(19) United States
(12) Patent Application Publication
(10) Pub. No.: US 2013/0052246 A1
(43) Pub. Date: Feb. 28, 2013

(54) COSMETIC COMPOSITION COMPRISING BORON NITRIDE COMPLEX POWDER

(71) Applicants: Hisao Ito, Tokyo (JP); Hideyuki Yamaki, Tokyo (JP); Naoyuki Enomoto, Fukuoka (JP)

(72) Inventors: Hisao Ito, Tokyo (JP); Hideyuki Yamaki, Tokyo (JP); Naoyuki Enomoto, Fukuoka (JP)

(73) Assignee: CHANEL PARFUMS BEAUTE, Neuilly Sur Seine (FR)

(21) Appl. No.: 13/696,456

(22) PCT Filed: Aug. 12, 2010
(86) PCT No.: PCT/EP2010/061787

§ 371 Date: Nov. 6, 2012

(30) Foreign Application Priority Data
May 7, 2010 (JP) 2010-106967

(51) Int. Cl. A61K 8/19; A61Q 1/10; A61K 8/27; A61Q 1/06; A61K 8/29; A61K 8/02; A61Q 1/02

(52) U.S. Cl. 424/401; 424/70.7; 424/63; 424/64

(57) ABSTRACT

A cosmetic composition includes a boron nitride complex powder in which particles of metal oxide are attached to at least a part of the surface of a base material consisting of boron nitride. The metal oxide is preferably a combination of titanium dioxide, yellow iron oxide, red iron oxide and black iron oxide. Preferred compositions include liquid or powder foundation, lipstick, eye shadow, eye liner and mascara. The cosmetic composition including a cosmetic powder is capable of imparting smoothness to the touch, lively finish with fine texture and natural glow, and adhesion to the skin when applied to the skin.

### TECHNICAL FIELD

The present invention relates to a cosmetic composition comprising a boron nitride complex powder, and in particular, a makeup cosmetic composition. More specifically, the present invention relates to a cosmetic composition, in which a boron nitride complex powder—which may provide smoothness to the touch, lively finish with fine texture and natural glow, and adhesion to the skin when applied to the skin—is introduced as an essential component.

### BACKGROUND ART

In general, in makeup cosmetics such as foundations, flake-like powder of mica, talc, kaolin or the like is introduced in order to improve adhesion to the skin. Further, in order to adjust color tone, white pigments such as titanium oxide and color pigments such as iron oxide are used. By adding titanium oxide, color unevenness of the skin such as spots and freckles can be covered. However, in this case, the skin may look dull, and a transparent and natural finish cannot be easily provided. In addition, since sufficient smoothness and gloss cannot be obtained, the unevenness of the skin cannot be evenly covered.

To resolve this problem, Japanese Laid-Open Patent Publication No. 2002-154929 for example suggested blending a first complex powder in which the surface of a flake-like powder is coated with spherical powder consisting of an organic polymer and a second complex powder in which the surface of a pearlescent pigment is coated with a color pigment. It has been reported that, according to this method, defects of the skin could be covered to provide naturally transparent and glossy finish. However, this method is complicated because 2 types of complex powders must be prepared and blended together.

Further, Japanese Laid-Open Patent Publication No. 2002-3744 discloses the use of a sintered complex pigment obtained by mixing and calcining a flake-like powder of titanated mica, sericite or the like, a particulate iron oxide and a particulate powder of titanium oxide or the like to provide adhesion to the skin and suitable gloss. However, when black iron oxide, yellow iron oxide or the like are used as the aforementioned particulate iron oxide, calcination oxidizes those iron oxide, which results in hue change. It becomes therefore difficult to control the hue to a desired degree.

Japanese Laid-Open Patent Publication No. 2000-86210 discloses a complex particle in which the surface of a metal oxide such as cerium oxide is coated with boron nitride. However, this document only studies the effect as an ultraviolet screening, but does not disclose the effect of these particles on the adhesion to the skin, well finish or feeling of glow.

### PRIOR ART DOCUMENTS

### Patent Documents

[Patent Document 1] Japanese Laid-Open Patent Publication No. 2002-154929

[Patent Document 2] Japanese Laid-Open Patent Publication No. 2002-3744

[Patent Document 3] Japanese Laid-Open Patent Publication No. 2000-86210

### DISCLOSURE OF THE INVENTION

### Problems to be Solved by the Invention

In view of the above, it is desirable to provide a novel cosmetic powder capable of imparting smoothness to the touch, lively finish with fine texture and natural glow, and adhesion to the skin when applied to the skin, as well as a cosmetic composition comprising said powder.

### Means for Solving the Problems

The present inventors diligently made researches in order to solve the above-described problem, and found that a good balance between smoothness to the touch and the covering ability for covering defects of the skin such as spots and wrinkles, lively finish with fine texture and natural glow, and adhesion to the skin could be provided by blending a complex powder in which particles of a metal oxide are attached to the surface of a base material consisting of boron nitride in a cosmetic composition such as a foundation. Thus, the present invention was achieved.

Therefore, the present invention relates to a cosmetic composition in which a boron nitride complex powder is blended as further described below.

In particular, the present invention relates to a cosmetic composition comprising a boron nitride complex powder in which particles of a metal oxide (hereinafter also referred to as “metal oxide particle”) are attached to at least a part of the surface of a base material consisting of boron nitride (hereinafter also referred to as “boron nitride base material”).

[2] The cosmetic composition according to item [1], wherein the metal oxide is one or a combination of two or more substances selected from the group consisting of titanium dioxide, iron oxide, zinc oxide and chrome oxide.

[3] The cosmetic composition according to item [1] or [2], wherein the metal oxide comprises a combination of titanium dioxide, yellow iron oxide, red iron oxide and black iron oxide.

[4] The boron nitride complex powder according to item [3], wherein the metal oxide comprises 50 to 95.8 wt % of titanium dioxide, 3 to 35 wt % of yellow iron oxide, 1 to 11 wt % of red iron oxide and 0.2 to 4 wt % of black iron oxide based on the total weight of the metal oxide.

[5] The cosmetic composition according to any one of items [1] to [4], wherein the average particle size of the boron nitride complex powder is 2 μm to 35 μm when measured by laser diffraction scattering method.

[6] The cosmetic composition according to any one of items [1] to [5], wherein the average particle size of the boron nitride base material is 1 μm to 30 μm when measured by laser diffraction scattering method, and wherein the average particle size of the particles of the metal oxide is 0.1 μm to 2 μm when measured with a scanning electron micrograph.

[7] The cosmetic composition according to any one of items [1] to [6], wherein the weight ratio of boron nitride base material/metal oxide particle is 75/25 to 30/70.

[8] The cosmetic composition according to any one of items [1] to [7], further comprising a component allowed to be used in cosmetics.

[9] The cosmetic composition according to any one of items [1] to [8], which comprises 0.1 to 50 wt % of the boron nitride complex powder based on the total weight of the cosmetic composition.

[10] The cosmetic composition according to any one of items [1] to [9], which is in the form of a makeup cosmetic or skincare cosmetic.

[11] The cosmetic composition according to item [10], which is in the form of a liquid foundation or powder foundation.

[12] The cosmetic composition according to item [10], which is in the form of a lipstick, eye shadow, eye liner or mascara.

### Advantageous Effect of the Invention

By using the cosmetic composition of the present invention, the balance between smoothness to the touch and the covering ability is improved. Moreover, when the cosmetic composition is applied to the skin, lively finish with fine texture and natural glow and adhesion to the skin can be provided.

### BRIEF DESCRIPTION OF THE DRAWINGS

FIG. 1 shows a microscope photograph of the boron nitride complex powder obtained in Production Example 1, which is used in the cosmetic composition of the present invention.

### BEST MODE FOR CARRYING OUT THE INVENTION

The boron nitride complex powder and the cosmetic composition in which it is blended will be hereinafter described in detail.

The boron nitride complex powder used in the present invention is a complex powder of a boron nitride base material and a metal oxide particle in which particles of a metal oxide are attached to at least a part of the surface of a base material consisting of boron nitride. The term “Attached” as used herein is intended to mean a state in which the boron nitride base material and the metal oxide particle are adhered to each other by the mutual intermolecular force. This should be distinguished from a “sintered body” in which solid particles are bound together by solid reaction. Therefore, sintered complex powders described in Japanese Laid-Open Patent Publication No. 2002-3744, should be excluded from the aforementioned boron nitride complex powder. In the boron nitride complex powder, the boron nitride base material and the metallic compound particle are preferably bound together by van der Waals' force.

The boron nitride base material used in the boron nitride complex powder is a white powder having flake-like crystal structure. There are hexagonal boron nitride and cubic boron nitride. In the present invention, a base material made of hexagonal boron nitride is preferably used. Examples of boron nitride base materials preferably used in the present invention include cosmetic boron nitrides of “SHP” series manufactured by Mizushima Ferroalloy Co., Ltd., and cosmetic boron nitrides of “BORONEIGE®” series manufactured by ESK Ceramics GmbH & Co. KG.

The particle size of the boron nitride base material is not particularly limited, and can suitably be selected depending on the purpose and intended use. However, in a preferred embodiment, the boron nitride base material is pulverized or shredded for use so that the average particle size (measured by the laser diffraction scattering method) is 1 μm to 30 μm. More preferably, the average particle size of the boron nitride base material used in the present invention is 2 μm to 20 μm, and even more preferably 5 μm to 10 μm. In this regard, without willing to be bound by any theory, it appears that when the average particle size is less than 1 μm, because of strong cohesion force, smoothness to the touch due to a boron nitride material may not be obtained. On the other hand, when the average particle size is more than 30 μm, unnatural and dull glow might be generated and lively finish may not be obtained.

In the present invention, the metal oxide particle attached to the surface of the boron nitride base material is not particularly limited. Those used as a white pigment or color pigment introduced in a makeup cosmetic composition can suitably be used. Specific examples thereof include titanium dioxide, iron oxide (including yellow iron oxide (iron oxyhydroxide and ferric hydroxide), red iron oxide (iron sesquioxide) and black iron oxide (ferrosoferric oxide)), zinc oxide, chrome oxide and mixtures thereof.

One or a combination of two or more types of metal oxide particles may be used. In a preferred embodiment, the metal oxide is one or a combination of two or more substances selected from the group consisting of titanium dioxide, iron oxide, zinc oxide and chrome oxide

In a more preferred embodiment, the metal oxide is a combination of titanium dioxide and iron oxide is preferred. In a further preferred embodiment, the metal oxide is a combination of titanium dioxide, yellow iron oxide, red iron oxide and black iron oxide.

The content of titanium dioxide is preferably 50 to 96 wt %, more preferably 57 to 92 wt %, and even more preferably 64 to 88 wt % based on the total weight of the metal oxide.

Further, the content of iron oxide is preferably 4 to 50 wt %, more preferably 8 to 43 wt %, and even more preferably 12 to 36 wt % based on the total weight of the metal oxide.

In the iron oxide, the content of yellow iron oxide is preferably 3 to 35 wt %, more preferably 6 to 30 wt %, and even more preferably 8 to 25 wt % based on the total weight of the metal oxide

Further, the content of red iron oxide is preferably 1 to 11 wt %, more preferably 2 to 10 wt %, and even more preferably 3 to 9 wt % based on the total weight of the metal oxide.

Further, the content of black iron oxide is preferably 0.2 to 4 wt %, more preferably 0.5 to 3 wt %, and even more preferably 1 to 2 wt % based on the total weight of the metal oxide.

In a preferred embodiment, when using a combination of titanium dioxide, yellow iron oxide, red iron oxide and black iron oxide, the metal oxide comprises 50 to 95.8 wt % of titanium dioxide, 3 to 35 wt % of yellow iron oxide, 1 to 11 wt % of red iron oxide and 0.2 to 4 wt % of black iron oxide.

In a more preferred embodiment, the metal oxide comprises 57 to 91.5 wt % of titanium dioxide, 6 to 30 wt % of yellow iron oxide, 2 to 10 wt % of red iron oxide and 0.5 to 3 wt % of black iron oxide.,

In an even more preferred embodiment, the metal oxide comprises 64 to 88 wt % of titanium dioxide, 8 to 25 wt % of yellow iron oxide, 3 to 9 wt % of red iron oxide and 1 to 2 wt % of black iron oxide.

When using the metal oxide within the above-described range, a good balance between hiding power and hue is provided. In addition, defects of the skin can be minimized at the time of application to the skin, and a glow can be added to the skin.

The particle size of the metal oxide particle to be used in the present invention is suitably selected depending on the purpose and intended use. However, the average particle size is preferably 0.1 μm to 2 μm, more preferably 0.2 μm to 1 μm, and even more preferably 0.3 μm to 0.8 μm when measured based on a scanning electron micrograph. In this regard, without willing to be bound by any theory, it appears that when the average particle size is less than 0.1 μm, the coloring power as a pigment may be reduced, rough feeling due to the particles may be increased and smoothness to the touch may not be obtained. On the other hand, when the average particle size is more 2 μm, coloring power as a pigment may reduced, but also the texture may become rough.

In a preferred embodiment of the present invention, the boron nitride complex powder is adjusted to have a desired size by suitably selecting the sizes of the boron nitride base material and the metal oxide particle to be used. In a preferred embodiment, when the boron nitride complex powder is introduced in the cosmetic composition of the present invention, its average particle size is preferably 2 μm to 35 μm when measured by the laser diffraction scattering method. More preferably, average particle size of the boron nitride complex powder is 3 μm to 25 μm, and even more preferably 6 μm to 15 μm. When the average particle size is less than 2 μm, due to strong cohesion force, it can result in little smoothness and gloss as well as reduced smoothness to the touch. On the other hand, when the average particle size is more than 35 μm, not only may adhesion to the skin be reduced, but also the texture may become rough. It is preferred that, after a boron nitride complex dry powder is prepared, the boron nitride complex powder be shredded or pulverized to provide a state in which complex powders are not aggregated together.

In a more preferred embodiment, when the boron nitride complex powder is introduced in the cosmetic composition of the present invention, the average particle size of boron nitride base material is 1 μm to 30 μm when measured by the laser diffraction scattering method and the average particle size of particles of metal oxide is 0.1 μm to 2 μm when measured by the laser diffraction scattering method.

In a preferred embodiment of the present invention, the size ratio between the boron nitride base material and the metal oxide particle (the average particle size of the former is represented by A; the average particle size of the latter is represented by B; and the particle size ratio is represented by A/B) is preferably 1 to 300, more preferably 2 to 100, and even more preferably 6 to 33.

In the present invention, values of the average particle size of the boron nitride complex powder, boron nitride base material or metal oxide particle are obtained using the measurement methods described below.

The weight ratio between the boron nitride base material and the metal oxide particle to be used in the boron nitride complex powder (boron nitride base material/metal oxide particle) varies depending on the type (e.g., specific gravity) of metal oxide used.

In a preferred embodiment of the present invention, the weight ratio between the boron nitride base material/metal oxide particle is 75/25 to 30/70.
In a more preferred embodiment, the weight ratio between the boron nitride base material /metal oxide particle is 70/30 to 35/65, and even more preferably 65/35 to 40/60. When the weight ratio is less than 30/70 (that is, the amount of the metal oxide is large), an aggregate of particles of the metal oxide which have failed to attach to the boron nitride base material may be generated. Without willing to be bound by this theory, rough feeling due to the aggregated particles may be increased, and smoothness to the touch as well as feeling of glow may not be obtained. On the other hand, when the weight ratio is more than 75/25 (that is, the amount of the metal oxide is small), sufficient coloring power may not be obtained, and desired effects may not be obtained due to too much feeling of glow.

In the boron nitride complex powder to be used in the present invention, it is sufficient when the metal oxide particle is attached to at least a part of the surface of the boron nitride base material. The entire surface of the boron nitride base material may be covered with the metal oxide particle, and there may also be a case where a part of the surface of the boron nitride base material is not covered with the metal oxide particle.

Methods for attaching the metal oxide particle to the surface of the boron nitride base material in the boron nitride complex powder may be any conventionally known methods as described below.

(1) A first method can be the following: the metal oxide particles are dispersed in a dispersion medium of water, alcohol or a mixture thereof to form a slurry. A suspension (usually a water suspension) of the boron nitride base material is also prepared. The slurry is then added to the suspension to be dispersed therein. Due to the electrostatic interaction therebetween, the metal oxide particles are attached to the surface of the boron nitride base material; and after that, the dispersion medium is separated and drying is performed.

In the method described in (1) above, the concentration of the solid content included in the slurry (i.e. the total amount of the metal oxide particle and the boron nitride base material) is preferably in the range of 5 to 50 wt %, and more preferably in the range of 10 to 30 wt % based on the total amount of the slurry.

(2) A second method can be the following: the metal oxide particles are dispersed in a dispersion medium of water, alcohol or a mixture thereof to form a slurry. A suspension (usually a water suspension) of the boron nitride base material is also prepared. The slurry is then added to the suspension to be dispersed therein; and the resulting dispersion liquid is sprayed into warm air from a spray drier or hot air streaming, thereby performing drying.

In the method described in (2) above, the concentration of the solid content included in the slurry (i.e. the total amount of the metal oxide particle and the boron nitride base material) is preferably in the range of 5 to 50 wt %, and more preferably 10 to 30 wt % based on the total weight of the slurry.

In each of the above-described methods, it is desired that the boron nitride base material on which the metal oxide particles are attached, is dried under the temperature condition of 150° C. or lower. More specifically, it is preferred that the above-described base material is dried under ordinary pressure or reduced pressure at 60 to 130° C., and preferably 80 to 110° C. for 0.1 to 20 hours. Indeed, when heating is conducted at more than 150° C.—though the situation also varies depending on heating time—and when using an iron oxide particle (in particular, black iron oxide particle, yellow iron oxide particle or the like as the aforementioned metal oxide particle), the iron oxide particles can be oxidized, therefore inducing a hue change. It may thus be difficult to control the hue to a desired degree. In this respect, when employing the aforementioned spray drying method, the drying temperature using a spray drier (temperature of air streaming at the inlet side) should preferably retained in the range of 60 to 130° C. When the drying time is less than 0.1 hour, drying may not be sufficiently performed, and when such a product is later introduced in a cosmetic, sense of use may be degraded and stability may be altered. It is further not preferable that the aforementioned drying time be more than 20 hours, since productivity would therefore be reduced.

In the dry powder obtained with the above-described methods (i.e., the boron nitride complex powder in which the aforementioned metal oxide particle is attached to the surface of the aforementioned boron nitride base material), base materials may be attached or aggregated together. Therefore, it is desired that the dry powder be shredded or pulverized using a mortar, atomizer, mixer or the like according to need and thereafter used in the next step.

The surface of the boron nitride complex powder used in the present invention may be subjected to a hydrophobization treatment. For example, the surface may be subjected to coating treatment using a metal soap of aluminum stearate, zinc stearate or the like; coating treatment using N-acylamino acid metal salt such as aluminum N-stearoyl glutamate and aluminum N-lauroyl glutamate; coating treatment using phospholipid such as lecithin; treatment of baking silicone such as hydrogen methyl polysiloxane and dimethyl polysiloxane; and silylation treatment using a silane coupling agent such as dimethoxydimethylsilane and methoxytrimethylsilane.

Components other than the metal oxide particle may be attached to the boron nitride complex powder used in the present invention, as long as the purpose and effects of the present invention are not altered. For example, compounds used in the above-described hydrophobization treatment may be attached to the complex powder.

The boron nitride complex powder used in the present invention is preferably a cosmetic powder since it can provide a good balance between smoothness and gloss and covering ability. When applied to the skin, smoothness to the touch is provided and at the same time, defects of the skin such as wrinkles and spots are covered. The boron nitride complex powder can also provide lively finish with fine texture, natural glow and adhesion to the skin.

The content of boron nitride complex powder in the cosmetic composition of the present invention varies depending on the purpose and intended use. In a preferred embodiment, the cosmetic composition of the present invention comprises 0.1 to 50 wt % of boron nitride complex powder, preferably 0.5 to 45 wt %, and more preferably 1 to 40 wt % based on the total weight of the cosmetic composition.

For example, when the cosmetic composition of the present invention is a powder foundation, the content of the boron nitride complex powder is preferably 0.1 to 50 wt %, more preferably 0.5 to 45 wt %, and even more preferably 1 to 40 wt % based on the total weight of the cosmetic composition. Without willing to be bound by any theory, it seems that when the content is less than the above-described ranges, the characteristics of the boron nitride complex powder might not be easily exerted. On the other hand, when the content is more than the above-described one, the covering ability of the boron nitride complex powder may become too strong, leading to unnatural finish.

Further, for example, when the cosmetic composition of the present invention is a liquid foundation, the content of the boron nitride complex powder is preferably 0.1 to 40 wt %, more preferably 0.5 to 35 wt %, and even more preferably 1 to 30 wt % based on the total weight of the cosmetic composition. Without willing to be bound by any theory, it seems that when the content is less than the above-described one, the characteristics of the boron nitride complex powder might not be easily exerted. On the other hand, when the content is more than the above-described one, the covering ability of the boron nitride complex powder may become too strong, leading to unnatural finish.

Further, for example, when the cosmetic composition of the present invention is an eye shadow, the content of the boron nitride complex powder is preferably 0.1 to 50 wt %, more preferably 1 to 45 wt %, and even more preferably 2 to 40 wt % based on the total weight of the cosmetic composition. Without willing to be bound by any theory, it seems that when the content is less than the above-described one, the characteristics of the boron nitride complex powder might not be easily exerted. On the other hand, when the content is more than the above-described one, effects corresponding to a blending amount may not be easily exerted.

Further, for example, when the cosmetic composition of the present invention is a blush, the content of the boron nitride complex powder is preferably 0.1 to 40 wt %, more preferably 0.5 to 35 wt %, and even more preferably 1 to 30 wt % based on the total weight of the cosmetic composition. Without willing to be bound by any theory, it seems that when the content is less than the above-described one, the characteristics of the boron nitride complex powder might not be easily exerted. On the other hand, when the content is more than the above-described one, effects corresponding to a blending amount may not be easily exerted.

Further, for example, when the cosmetic composition of the present invention is a mascara, the content of the boron nitride complex powder is preferably 0.5 to 35 wt %, more preferably 1 to 30 wt %, and even more preferably 2 to 25 wt % based on the total weight of the cosmetic composition. Without willing to be bound by any theory, it seems that when the content is less than the above-described one, the characteristics of the boron nitride complex powder might not be easily exerted. On the other hand, when the content is more than the above-described one, effects corresponding to a blending amount may not be easily exerted.

Further, for example, when the cosmetic composition of the present invention is a lipstick or lip gloss, the content of the boron nitride complex powder is preferably 0.1 to 35 wt %, more preferably 0.5 to 30 wt %, and even more preferably 1 to 25 wt % based on the total weight of the cosmetic composition. Without willing to be bound by any theory, it seems that when the content is less than the above-described one, the characteristics of the boron nitride complex powder might not be easily exerted. On the other hand, when the content is more than the above-described one, effects corresponding to a blending amount may not be easily exerted.

Further, for example, when the cosmetic composition of the present invention is a lipliner, the content of the boron nitride complex powder is preferably 0.1 to 30 wt %, more preferably 0.5 to 25 wt %, and even more preferably 1 to 20 wt % based on the total weight of the cosmetic composition. Without willing to be bound by any theory, it seems that when the content is less than the above-described one, the characteristics of the boron nitride complex powder might not be easily exerted. On the other hand, when the content is more than the above-described one, effects corresponding to a blending amount may not be easily exerted.

Further, for example, when the cosmetic composition of the present invention is a concealer, the content of the boron nitride complex powder is preferably 0.1 to 40 wt %, more preferably 1 to 35 wt %, and even more preferably 2 to 30 wt % based on the total weight of the cosmetic composition. Without willing to be bound by any theory, it seems that when the content is less than the above-described one, the characteristics of the boron nitride complex powder might not be easily exerted. On the other hand, when the content is more than the above-described one, effects corresponding to a blending amount may not be easily exerted.

Further, for example, when the cosmetic composition of the present invention is an eyeliner, the content of the boron nitride complex powder is preferably 0.1 to 30 wt %, more preferably 0.5 to 25 wt %, and even more preferably 1 to 20 wt % based on the total weight of the cosmetic composition. Without willing to be bound by any theory, it seems that when the content is less than the above-described one, the characteristics of the boron nitride complex powder might not be easily exerted. On the other hand, when the content is more than the above-described one, effects corresponding to a blending amount may not be easily exerted.

Further, for example, when the cosmetic composition of the present invention is an eyeliner pencil, the content of the boron nitride complex powder is preferably 0.5 to 50 wt%, more preferably 1 to 45 wt%, and even more preferably 2 to 35 wt% based on the total weight of the cosmetic composition. Without willing to be bound by any theory, it seems that when the content is less than the above-described one, the characteristics of the boron nitride complex powder might not be easily exerted. On the other hand, when the content is more than the above-described one, effects corresponding to a blending amount may not be easily exerted.

In addition to the boron nitride complex powder, the cosmetic composition of the present invention may preferably further comprise components allowed to be used in cosmetics within a range such that the purpose and effects of the present invention are not reduced.

Examples of components allowed to be used in cosmetics include powder components other than the boron nitride complex powder of the present invention, a liquid oil, a solid fat, a wax, a hydrocarbon, a higher fatty acid, a higher alcohol (preferably an alcohol having 6 or more carbon atoms, and more preferably an alcohol having 10 or more carbon atoms), a synthetic ester oil, a silicone oil, a surfactant, a co-surfactant, a moisturizing agent, a film forming agent, a thickener, a gelling agent, an inorganic mineral, a metal sequestering agent, a lower alcohol, a polyhydric alcohol, a monosaccharide, an oligosaccharide, an amino acid, a plant extract, an organic amine, a polymer emulsion, an antioxidant, an antioxidant aid, a skin nutrient, a vitamin, a blood flow promoter, an antibacterial agent, an anti-inflammatory agent, a cell (skin) activating agent, a keratolytic agent, a refrigerant, a water-soluble polymer, a skin whitening agent, a UV absorber, an anti-fading agent, an antiseptic agent, a skin softener, an antiaging agent, an anti-pollution agent, a keratolytic agent, a pH adjustor, a buffer, a perfume and water.

Any of the above-described components may be suitably selected and blended depending on a desired formulation and product form. The blending amount of these additional components is not particularly limited as long as it does not alter the purpose of the present invention. The blending amount is suitably selected depending on a formulation, product form, etc.

Examples of powder components include inorganic powders, such as talc, kaolin, mica, sericite, white mica, gold mica, a synthetic mica, red mica, black mica, vermiculite, magnesium carbonate, calcium carbonate, aluminum silicate, barium silicate, calcium silicate, magnesium silicate, strontium silicate, a metal tungstate, silica, zeolite, barium sulfate, magnesium sulfate, calcined calcium sulfate (calcined gypsum), calcium phosphate, fluorine apatite, hydroxyapatite, ceramic powder, metallic soap (e.g. zinc myristate, calcium palmitate, aluminum stearate and magnesium stearate) and boron nitride; organic powders, such as polyamide resin powder (nylon powder), polyethylene powder, polymethyl methacrylate powder, polystyrene powder, styrene/acrylic acid copolymer resin powder, benzoguanamine resin powder, polytetrafluoroethylene powder and cellulose powder; metal powder pigments, such as aluminum powder and copper powder; organic pigments, such as a zirconium-, barium-, and aluminum-lakes; and natural colors, such as chlorophyll and β-carotene. Note that the powder components may be hydrophobized.

Examples of liquid oil include avocado oil, camellia oil, turtle oil, macadamia nut oil, corn oil, mink oil, olive oil, rapeseed oil, yolk oil, sesame oil, persic oil, wheat germ oil, camellia kissi oil, castor oil, linseed oil, safflower oil, cotton seed oil, perilla oil, soybean oil, peanut oil, tea seed oil, Torreya seed oil, rice bran oil, Chinese tung oil, Japanese tung oil, jojoba oil, germ oil, and triglycerin.

Examples of solid fat include cacao butter, coconut oil, horse tallow, hardened coconut oil, palm oil, palm kernel oil, Japan tallow kernel oil, hardened oil, Japan tallow, and hardened castor oil.

Examples of wax include bees wax, candelilla wax, cotton wax, carnauba wax, bayberry wax, Chinese insect wax, montan wax, bran wax, lanolin, kapok wax, acetylated lanolin, liquid lanolin, sugar cane wax, lanolin fatty acid isopropyl ester, hexyl laurate, reduction lanolin, jojoba wax, hard lanolin, shellac wax, POE lanolin alcohol ether, POE lanolin alcohol acetate, POE cholesterol ether, lanolin fatty acid polyethylene glycol, and POE hydrogenated lanolin alcohol ether.

Examples of hydrocarbon oil include liquid paraffin, ozokerite, squalane, pristane, paraffin, ceresin, squalene, vaseline, microcrystalline wax, and hydrogenated polydecene.

Examples of higher fatty acid include lauric acid, myristic acid, palmitic acid, stearic acid, behenic acid, oleic acid, undecylenic acid, tall oil acid, isostearic acid, linoleic acid, linolenic acid, eicosapentaenoic acid (EPA), and docosahexaenoic acid (DHA).

Examples of higher alcohol include linear alcohols, such as lauryl alcohol, cetyl alcohol, stearyl alcohol, behenyl alcohol, myristyl alcohol, oleyl alcohol, and cetostearyl alcohol; branched alcohols, such as monostearyl glyceryl ether (batyl alcohol), 2-decyltetradecanol, lanolin alcohol, cholesterol, phytosterol, hexyldodecanol, isostearyl alcohol, and octyldodecanol.

Examples of synthetic ester oil include tripropylene glycol dineopentanoate, isononyl isononanoate, isotridecyl isononanoate, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, acetylated lanolin, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprylate, diisostearyl malate, glyceryl di-2-heptylundecanoate, trimethylolpropane tri-2-ethylhexanoate, trimethylolpropane triisostearate, pentaerythritol tetra-2-ethylhexanoate, glyceryl tri-2-ethylhexanoate, glyceryl trioctanoate, glyceryl triisopalmitate, trimethylolpropane triisostearate, cetyl 2-ethylhexanoate, 2-ethylhexyl palmitate, glyceryl trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, bis-behenyl/isostearyl/phytosteryl dimer dilinoleyl dimer dilinoleate, phytosteryl/behenyl/octyldodecyl/isostearyl lauroyl glutamate, and tri(caprylic acid/capric acid) glyceryl.

Examples of silicone oil include a chain polysiloxane, such as dimethicone, methyl trimethicone, methylphenylpolysiloxane and diphenylpolysiloxane; a cyclic polysiloxane, such as octamethyl cyclotetrasiloxane, decamethyl cyclopentasiloxane and dodecamethyl cyclohexasiloxane; a silicone resin forming a 3D net structure; a silicone rubber; various modified polysiloxanes, such as amino-modified polysiloxane, polyether-modified polysiloxane, alkyl-modified polysiloxane and fluorine-modified polysiloxane.

Examples of silicone elastomers include non-emulsifying organopolysiloxane elastomers or emulsifying organosiloxane elastomers. Examples of the non-emulsifying organopolysiloxane elastomers include dimethicone/vinyl dimethicone crosspolymers, lauryl dimethicone/vinyl dimethicone crosspolymers, and the like.

The dimethicone/vinyl dimethicone crosspolymers include products commercially available from DOW CORNING, Midland, Mich., under the trade name of, for example, DC 9040 and DC 9045; products commercially available from MOMENTIVE under the trade name of SFE 839 and the Velvasil series products; products commercially available from SHIN ETSU under the trade name of, for example, KSG-15, KSG-16, and KSG-18 ([dimethicone/phenyl vinyl dimethicone crosspolymer]); and Gransil™ series products from GRANT INDUSTRIES.

The lauryl dimethicone/vinyl dimethicone crosspolymers include products commercially available from SHIN ETSU under the trade name of, for example, KSG-31, KSG-32, KSG-41, KSG-42, KSG-43, and KSG-44.

Examples of emulsifying organosiloxane elastomers include polyalkoxylated silicone elastomers, polyglycerolated silicone elastomers, or the like.

The polyalkoxylated silicone elastomers include products commercially available from DOW CORNING under the trade name of, for example, DC9010 and DC9011; products commercially available from SHIN ETSU under the trade name of, for example, KSG-20, KSG-21, KSG-30, KSG-31, KSG-32, KSG-33, KSG-210, KSG-310, KSG-320, KSG-330, KSG-340, and X-226146.

The polyglycerolated silicone elastomers include products commercially available from SHIN ETSU under the trade name of, for example, KSG-710, KSG-810, KSG-820, KSG-830, KSG-840, KSG-31, KSG-32, KSG-41, KSG-42, KSG-43, and KSG-44. In addition, examples of silicone elastomers into which 2 types of branches, i.e., a silicone chain and an alkyl chain have been introduced include products commercially available from SHIN ETSU under the trade name of, for example,

KSG-042Z, KSG-045Z, KSG-320Z, KSG-350Z, KSG-820Z, and KSG-850Z.

Silicone elastomers comprising a polyalkyl ether group as pendant or cross linked may also included as components in the cosmetic composition of the present invention. Particularly suitable silicone elastomers comprising a polyalkyl ether group include compounds with the international Nomenclature of Cosmetic Ingredients (INCI) name: bis-vinyldimethiconlbis-isobutyl PPG-20 crosspolymer, bis-vinyldimethicone/PPG-20 crosspolynier, dimethicone/bis-isobutyl PPG-20 crosspolymer, dimethicone:/PPG-20 crosspolymer, and dimethicone/bis-secbutylPPG-20crosspolymer. Such cross-linked elastomers are available from Dow Corning under the experimental names of SOEB-1, SOEB-2, SOEB-3 and SOEB-4, and under the proposed commercial name of DC EL-8052 IH Si Organic Elastomer Blend, The elastomer particles are supplied pre-swollen in the respective solvents, isododecane (for SOEB 1-2), isohexadecane (for SOEB-3), and isodecyl neopentanoate (for SOEB-4).

Examples of surfactant include a lipophilic nonionic surfactant and a hydrophilic nonionic surfactant.

Examples of lipophilic nonionic surfactant include a sorbitan fatty acid ester, such as sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2-ethylhexylate and diglycerol sorbitan tetra-2-ethylhexylate; a glyceryl polyglyceryl fatty acid, such as glyceryl mono-cotton seed oil fatty acid, glyceryl monoerucate, glyceryl sesquioleate, glyceryl monostearate, glyceryl α,α′-oleate pyroglutamate, and glyceryl monostearate malate; a propylene glycol fatty acid ester such as monostearate propylene glycol; a hydrogenated castor oil derivative; and a glycerin alkyl ether.

Examples of hydrophilic nonionic surfactant include a POE-sorbitan fatty acid ester, such as POE-sorbitan monooleate, POE-sorbitan monostearate, POE-sorbitan monooleate and POE-sorbitan tetraoleate; a POE sorbitol fatty acid ester, such as POE-sorbitol monolaurate, POE-sorbitol monooleate, POE-sorbitol pentaoleate and POE-sorbitol monostearate; a POE-glycerin fatty acid ester, such as POE-glycerin monostearate, POE-glycerin monoisostearate and POE-glycerin triisostearate; a POE-fatty acid ester, such as POE-monooleate, POE-distearate, POE-monodioleate and ethylene glycol distearate; a POE-alkyl ether, such as POE-lauryl ether, POE-oleyl ether,

POE-stearyl ether, POE-behenyl ether, POE-2-octyldodecyl ether and POE-cholestanol ether; a Pluronic type surfactant (e.g., Pluronic); a POE-POP-alkyl ether, such as POE-POP-cetyl ether, POE-POP-2-decyltetradecyl ether, POE-POP-monobutyl ether, POE-POP-hydrogenated lanolin and POE-POP-glycerin ether.

Examples of co-surfactants include higher alcohols. Among them, linear alcohols such as lauryl alcohol, cetyl alcohol, stearyl alcohol, behenyl alcohol, myristyl alcohol, oleyl alcohol, cetostearyl alcohol, and the like, are preferable. Cetyl alcohol is particularly preferable.

Examples of metal sequestering agent include 1-hydroxyethane-1,1-diphosphonic acid; 1-hydroxyethane-1,1-diphosphonic acid tetrasodium salt; disodium edetate; trisodium edetate; tetrasodium edetate; sodium citrate; sodium polyphosphate; sodium metaphosphate; gluconic acid; phosphoric acid; citric acid; ascorbic acid; succinic acid; edetic acid; and trisodium ethylenediamine hydroxyethyl triacetate.

Examples of lower alcohol include ethanol, propanol, isopropanol, isobutyl alcohol, and t-butyl alcohol.

Examples of polyhydric alcohol include a dihydric alcohol, such as ethylene glycol, propylene glycol, pentylene glycol, trimethylene glycol, 1,2-butylene glycol, 1,3-butylene glycol, tetramethylene glycol, 2,3-butylene glycol, pentamethylene glycol, 2-butene-1,4-diol, hexylene glycol and octylene glycol; a trihydric alcohol, such as glycerin and trimethylolpropane; a tetrahydric alcohol such as pentaerythritol (e.g., 1,2,6-hexanetriol); a pentahydric alcohol such as xylitol; a hexahydric alcohol, such as sorbitol and mannitol; a polyhydric alcohol polymer, such as diethylene glycol, dipropylene glycol, triethylene glycol, polypropylene glycol and tetraethylene glycol; a dihydric alcohol alkyl ether, such as ethylene glycol monomethyl ether and ethylene glycol monoethyl ether; a dihydric alcohol alkyl ether, such as diethylene glycol monomethyl ether, diethylene glycol monoethyl ether and diethylene glycol monobutyl ether; a dihydric alcohol ether ester, such as ethylene glycol monomethyl ether acetate and ethylene glycol monoethyl ether acetate; a glycerol monoalkyl ether, such as chimyl alcohol, selachyl alcohol and batyl alcohol; and a sugar alcohol, such as sorbitol, maltitol, maltotriose, mannitol, sucrose, erythritol, glucose, fructose, starch sugar, maltose, xylitose, and a reduced alcohol of a starch sugar.

Examples of monosaccharide include a triose, such as D-glyceryl aldehyde and dihydroxyacetone; a tetrose, such as D-erythrose, D-erythrulose, D-threose and erythritol; a pentose, such as L-arabinose, D-xylose, L-lyxose, D-arabinose, D-ribose, D-ribulose, D-xylulose and L-xylulose; a hexose, such as D-glucose, D-talose, D-psicose, D-galactose, D-fructose, L-galactose, L-mannose and D-tagatose; a heptose, such as aldoheptose and heprose; an octose such as octurose; a deoxy sugar, such as 2-deoxy-D-ribose, 6-deoxy-L-galactose and 6-deoxy-L-mannose; an amino sugar , such as D-glucosamine, D-galactosamine, sialic acid, amino uronic acid and muramic acid; a uronic acid, such as D-glucuronic acid, D-mannuronic acid, L-guluronic acid, D-galacturonic acid and L-iduronic acid.

Examples of oligosaccharide include sucrose, lactose, maltose, trehalose, cellobiose, gentiobiose, umbilicin, raffinose, gentianose, maltotriose, melezitose, planteose, unbelliferose, stachyose, and verbascose.

Examples of amino acid include a neutral amino acid, such as threonine and cysteine; and a basic amino acid such as hydroxylysine. Further, as an amino acid derivative, for example, sodium acyl sarcosinate (sodium lauroyl sarcosinate), acyl glutamate, sodium acyl β-alanine, glutathione, and pyrrolidone carboxylic acid may be exemplified.

Examples of organic amine include monoethanolamine, diethanolamine, triethanolamine, morpholine, triisopropanolamine, 2-amino-2-methyl-1,3-propanediol, and 2-amino-2-methyl-1-propanol.

Examples of polymer emulsion include an acrylic resin emulsion, a poly(ethyl acrylate) emulsion, an acrylic resin solution, a poly(alkyl acrylate) emulsion, a poly(vinyl acetate) emulsion, and a natural rubber latex.

Examples of vitamin include vitamins A, B₁, B₂, B₆, C and E and derivatives thereof, pantothenic acid and derivatives thereof and biotin.

Examples of antioxidants include ascorbic acid and its derivatives such as ascorbyl palmitate, ascorbyl tetraisopalmitate, ascorbyl glucoside, magnesium ascorbyl phosphate, sodium ascorbyl phosphate and ascorbyl sorbate; tocopherol and its derivatives, such as tocopheryl acetate, tocopheryl sorbate, and other esters of tocopherol; dibutyl hydroxytoluene (BHT) and butylated hydroxyanisole (BHA); gallic acid ester; phosphoric acid; citric acid; maleic acid; malonic acid; succinic acid; fumaric acid; cephalin; a hexametaphosphate; phytic acid; ethylenediaminetetraacetic acid; and plant extracts, for instance from Chondrus cripsus, Rhodiola, Thermus thermophilus, mate leaves, oak wood, kayu rapet bark, sakura leaves and ylang ylang leaves.

Examples of moisturizing agent include polyethylene glycol; propylene glycol; dipropylene glycol; glycerin; 1,3-butylene glycol; xylitol; sorbitol; maltitol; mucopolysaccharides such as chondroitin sulfuric acid; hyaluronic acid; mucoitinsulfuric acid; caronic acid; atelo-collagen; cholesteryl-12-hydroxystearate; bile salt; a main component of NMF (natural moisturizing factor), such as a pyrrolidone carboxylic acid salt and a lactic acid salt; amino acids such as urea, cysteine and serine; short-chain soluble collagen; a diglycerin (EO) PO addition product; homo- and copolymers of 2-methacryloyloxyethylphosphorylcholine commercially available from NOF under the name of, for example, Lipidure HM and Lipidure PBM; panthenol; allantoin; PEG/PPG/Polybutylene Glycol-8/5/3 Glycerin commercially available from NOF under the trade name of Wilbride S 753; Trimethylglycine commercially available from Asahi KASEI Chemicals under the trade name of AMINOCOAT; and various plant extracts such as Castanea sativa extracts, hydrolyzed hazelnut proteins, Polianthes tuberosa polysaccharides, Argania spinosa kernel oil, and an extract of pearl containing conchiolin commercially available from Maruzen Pharmaceuticals under the trade name of Pearl Extract™.

Examples of skin softener include glyceryl polymethacrylate, methyl gluceth-20 and the like.

Examples of antiaging agent include acyl amino acids (specifically, products commercially available from SEDERMA under the trade name of Maxilip, Matrixyl 3000 or Biopeptide CL, or product commercially available from SEPPIC under the trade name of Sepilift); Pisum sativum extracts; hydrolyzed soy proteins; methylsilanol mannuronate; hydrolyzed cucurbita pepo seedcake; Scenedesmus extract; and the like.

Examples of anti-pollution agents include Moringa pterygosperma seed extracts (specifically, product commercially available from LSN under the trade name of Purisoft); Shea butter extract (specifically, products commercially available from SILAB under the trade name of Detoxyl, a blend of ivy extract, phytic acid and sunflower seed extract (for example, product commercially available from SEDERMA under the trade name of OSMOPUR), and the like.

Examples of keratolytic agents include α-hydroxy acids (specifically, glycolic, lactic, citric, malic, mandelic or tartaric acid), β-hydroxy acids (specifically, salicylic acid), esters thereof (specifically, C₁₂₋₁₃ alkyl lactate), and plant extracts containing these hydroxy acids (specifically, Hibiscus sabdriffa extracts), and the like.

Examples of water-soluble polymer include dextrin, methylcellulose, ethylcellulose, sodium carboxymethylcellulose, hydroxyethyl cellulose, hydroxypropyl cellulose, hydroxypropylmethylcellulose stearoyl ester, PVA, PVM, PVP, locust bean gum, guar gum, tara gum, tamarind gum, glucomannan, xylan, mannan and agar.

Examples of anti-inflammatory agents include bisabolol, allantoin, tranexamic acid, zinc oxide, sulfur oxide and its derivatives, chondroitin sulfate, and glycyrrhizinic acid and its derivatives (for example, glycyrrhizinates).

The cosmetic composition of the present invention may contain at least one whitening agent to block the synthesis of structural proteins such as the melanocyte-specific glycoprotein Pme117 involved in the mechanism of melanogenesis (stage I). Example of such a whitening agent may include the ferulic acid-containing cytovector (water, glycol, lecithin, ferulic acid, hydroxyethylcellulose) commercially available from BASF under the trade name of Cytovector™.

Furthermore, if necessary, the cosmetic composition of the present invention may contain at least one peptide as described in International Publication WO2009/010356 pamphlet.

Furthermore, if necessary, the cosmetic composition of the present invention may include a whitening agent having an inhibition effect on melanin synthesis and/or an inhibition effect on nanophthalmia-related transcription factor (MITF) expression and/or an anti-tyrosinase activity and/or an inhibition effect on endothelin-1 synthesis. Examples of such a whitening agent may include Glycyrrhiza glabra extract commercially available from Maruzen Pharmaceuticals under the trade name of Licorice extract™.

Furthermore, if necessary, the cosmetic composition of the present invention may include whitening agents having an antioxidant effect as well, such as vitamin C compounds, which include ascorbate salts, ascorbyl esters of fatty acids or of sorbic acid, and other ascorbic acid derivatives. Specific examples include ascorbyl phosphates (magnesium ascorbyl phosphate, sodium ascorbyl phosphate, and the like), and saccharide esters of ascorbic acid (ascorbyl-2-glucoside, 2-O-α-D-glucopyranosyl L-ascorbate, 6-O-β-D-galactopyranosyl L-ascorbate, and the like). Active agents of this type are commercially available from DKSH under the trade name of Ascorbyl glucoside™.

Furthermore, if necessary, the cosmetic composition of the present invention may include other whitening agents. Examples of the other whitening agents include pigmentation inhibiting agents such as plant extracts (e.g., Narcissus tazetta extracts), cetyl tranexamate (Nikko Chemicals Co., Ltd; trade name: NIKKOL TXC), arbutin, kojic acid, ellagic acid, cysteine, 4-thioresorcin, resorcinol or rucinol or their derivatives, glycyrrhizinic acid, hydroquinone-β-glucoside, and the like.

Furthermore, if necessary, the cosmetic composition of the present invention may also include organic and/or inorganic sunscreens.

Examples of organic sunscreens may include dibenzoylmethane derivatives such as butyl methoxydibenzoylmethane (product commercially available from HOFFMANN LA ROCHE under the trade name of Parsol 1789); cinnamic acid derivatives such as octyl methoxycinnamate (product commercially available from HOFFMANN LA ROCHE under the trade name of Parsol MCX), salicylates, para-aminobenzoic acids; β,β′-diphenylacrylate derivatives; benzophenone derivatives; benzylidenecamphor derivatives such as terephtalylidene dicamphor sulphonic acid; phenylbenzimidazole derivatives; triazine derivatives; phenylbenzotriazole derivatives; anthranilic acid derivatives, and the like, all of which may be coated or encapsulated.

Examples of inorganic sunscreens may include pigments and nanopigments formed from coated or uncoated metal oxides. Examples of the nanopigments include titanium oxide, iron oxide, zinc oxide, zirconium oxide and cerium oxide nanopigments, which are all well known as UV photoprotective agents.

Examples of antiseptic agent include p-hydroxybenzoate ester (e.g., methylparaben and propylparaben) and phenoxyethanol.

In addition, additives mentioned in International Cosmetic Ingredient Dictionary and Handbook, 13th Edition, 2010, published by the Personal Care Products Council, can be used in the cosmetic composition of the present invention.

The formulation of the cosmetic composition of the present invention is arbitrarily selectable, and a preferred formulation may be employed depending on a product form. For example, a solution, an emulsion, a dry powder dispersion, a water-oil double layer, a water-oil-powder triple layer, a gel and an oil can be employed.

The cosmetic composition of the present invention can be obtained by blending the boron nitride complex powder of the present invention and any optional components allowed to be used in cosmetics and mixing them using a method usually employed in preparation of a cosmetic composition. If necessary, the cosmetic composition of the present invention may be shaped.

The cosmetic composition of the present invention is preferably used in the form of makeup cosmetics (e.g., foundation, eye shadow, blush, mascara, lip makeup product, body makeup product, and nail product) and skincare cosmetics (e.g., emulsion, cream, and sunscreen). The cosmetic composition of the present invention is suitably used for makeup cosmetic compositions for the skin, eyelashes, eyebrows or lips, in particular, foundation (preferably powder foundation and liquid foundation), eye shadow, blush, mascara, lip makeup products (preferably lipstick, lip gloss and lipliner), concealer, eyeliner, eyeliner pencil, etc.

When preparing the above-described cosmetic composition, the boron nitride complex powder can either be blended to an existing conventional formulation, or introduced in replacement of a color material. Blending/formulation of cosmetic compositions is described in, for example, “Shin-Keshohin-gaku (New Cosmetology)”, Takeo Mitsui Ed., Nanzando Co., Ltd. (2nd edition, published on Jan. 18, 2001).

### EXAMPLES

The present invention will be more specifically described hereinafter by way of Examples and Comparative Examples. However, the present invention is not limited only to the Examples.

### 1. Preparation of Boron Nitride Complex Powder

### (1) Production Example 1

500 g of boron nitride having the average particle size of 7 μm (SHPT, Mizushima Ferroalloy Co., Ltd.) was added to 2833 ml of pure water, and the mixture was well stirred and heated to 60° C. PH of the mixture was then adjusted to 4.0 using hydrochloric acid having the concentration of 5 wt %, thereby obtaining a suspension.

Next, 402 g of titanium oxide particle having the average particle size of 0.3 μm (Tipaque CR-50, Ishihara Sangyo Kaisha Ltd.), 69 g of yellow iron oxide particle having the average particle size of 0.4 μm (TAROX LL-100P, Titan Kogyo, Ltd.), 23 g of red iron oxide particle having the average particle size of 0.4 μm (TAROX R-516P, Titan Kogyo, Ltd.), and 6 g of black iron oxide particle having the average particle size of 0.4 μm (TAROX BL-100P, Titan Kogyo, Ltd.) were added to 2833 ml of pure water, and well mixed to be homogeneously dispersed. The mixture was then added to the aforementioned suspension with stirring over 17 hours, and was cooled to room temperature. During the mixing step, pH of the suspension was retained at 4.0 using hydrochloric acid having the concentration of 1 wt %.

The obtained mixture was then cooled to room temperature, subjected to filtration and then dried at 110° C. for 16 hours.

Then, the obtained mass of dry powder was shredded (or pulverized) using a mixer, thereby obtaining 1000 g of boron nitride complex powder to which 4 types of metal oxide particles, i.e., the titanium oxide particle, yellow iron oxide particle, red iron oxide particle and black iron oxide particle were attached. The surface appearance of the powder was beige.

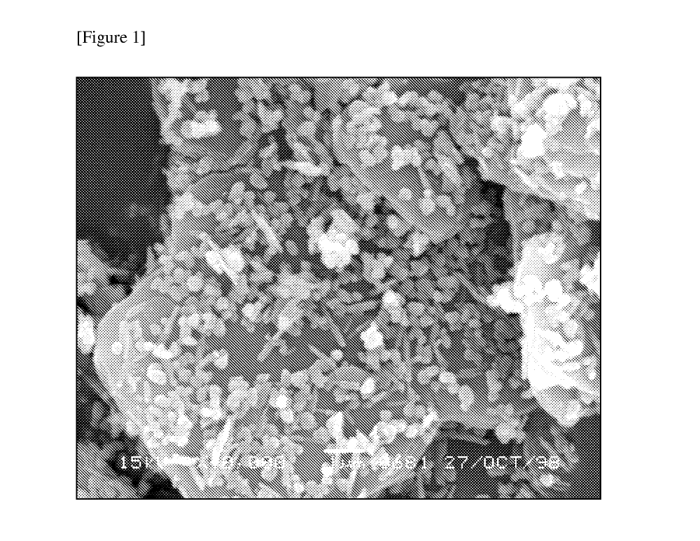

The average particle size of the boron nitride complex powder obtained in Production Example 1, measured using a particle size distribution measurement apparatus (LA-300, Horiba Ltd.) based on the laser diffraction scattering method, was 8 μm. FIG. 1 shows a micrograph (magnified 10,000 times) obtained using a scanning electron microscope (JSM-5600, JEOL). The color tone of the powder was measured using a spectrophotometric colorimeter (CM2600d, Konica Minolta Sensing, Inc.), and it was L*76, a*12, and b*18.

### (2) Production Example 2

1000 g of boron nitride complex powder obtained according to Production Example 1 was collected into a Henschel mixer (FM5C/I, Mitsui Mining Co., Ltd.). 20 g of methyl hydrogen polysiloxane (KF-9901, Shin-Etsu Chemical Co., Ltd.) was added thereto with stirring, and stirring was performed for 20 minutes.

The powder was then taken out from the Henschel mixer and dried at 110° C. for 15 hours, thereby obtaining a hydrophobized boron nitride complex powder which was surface-treated with methyl hydrogen polysiloxane (hydrogen dimethicone). The surface appearance of the powder was beige.

Further, the color tone of the powder was measured, and it was L*73, a*14, and b*23.

### (3) Production Example 3

A crucible was filled with 100 g of boron nitride complex powder obtained in Production Example 1, and calcination was performed using an electric furnace at 900° C. for 2 hours. Then cooling and pulverization were performed, thereby obtaining a complex sintered powder in which the metal oxide particles had been attached to the boron nitride powder and had been subjected to a calcination treatment. The surface appearance of the powder was red-brown. Further, the color tone of the powder was measured, and it was L*64, a*25, and b*20.

### [Measurement Methods]

The measurement methods employed in the Examples of the present invention will be further described in detail hereinafter.

### [Method for Measurement of the Average Particle Size]

### (1) Average Particle Size of Boron Nitride Base Material or Boron Nitride Complex Powder

The average particle size of a sample of the boron nitride base material or boron nitride complex powder was measured using a particle size distribution measurement apparatus (LA-300, Horiba Ltd.) based on the laser diffraction scattering method.

### (2) Average Particle Size of Titanium Oxide Particle

100 particles selected from the sample of the titanium oxide particle were photographed using a scanning electron microscope (S-5200N, Hitachi, Ltd.), and the average value of the particle size was measured.

### (3) Average Particle Size of Iron Oxide Particle

100 particles selected from the sample of the iron oxide particle were photographed using a scanning electron microscope (S-5200N, Hitachi, Ltd.), and the average value of the particle size (½ of the total value of the longer diameter and the shorter diameter) was measured.

### [Method for Measurement of Color Tone]

12 g of each of the complex powders of Production Examples 1-3 was collected into a round gold dish having the diameter of 58 mm (Miyoshi Kogyo Co., Ltd.). To prepare a measurement sample, pressing of the collected powders was performed for 1 second at a pressure of 0.2 MPa using a pressing machine (Pneumatic Power Cylinder Mini, Hirotaka Mfg. Co., Ltd.). For each sample, L*, a* and b* of the L*a*b* color system were measured according to the SCI method with the light source of D-65 and the visual field of 10° by using a spectrophotometric colorimeter (CM2600d, Konica Minolta Sensing, Inc.)

### 2. Preparation of Cosmetic Composition

Cosmetic compositions (i.e., a powder foundation, a liquid foundation, a lip stick and a mascara) having the below-described compositions, were prepared using the boron nitride complex powder obtained in Production Example 1 and the hydrophobized boron nitride complex powder obtained in Production Example 2. The methods of preparation of these cosmetic compositions are generally-used conventionally known methods. As a comparison, cosmetic compositions using a conventionally known cosmetic powder material and the boron nitride complex sintered powder obtained in Production Example 3 and having the below-described compositions were prepared in the same way.

### [1] Powder Foundation

The below-described components (1) to (14) were mixed together to obtain a first mixture. Components (15) to (18) were also mixed together to obtain a second mixture. the second mixture was then added to the first one and mixed. The obtained mixture was then pulverized using a hammer mill (Dalton Co., Ltd.), and weighed. We obtained 13 g of the pulverized mixture. The resulting pulverized mixture was then pressed using a powder pressing machine (Sanshinseiki Co., Ltd.) to obtain a powder foundation.

TABLE 1

| | | | | Comparative | Comparative |
|---|---|---|---|---|---|
| | | Example 1 | Example 2 | Example 1 | Example 2 |
| (1) | Hydrophobized titanium oxide* | 26.0 | 26.0 | 26.0 | 26.0 |
| (2) | Hydrophobized iron oxide* | 4.0 | 4.0 | 4.0 | 4.0 |
| (3) | Hydrophobized talc* | 10.0 | 10.0 | 10.0 | 10.0 |
| (4) | Hydrophobized mica* | 10.0 | 10.0 | 10.0 | 10.0 |
| (5) | Nylon powder** | 6.1 | 6.1 | 6.1 | 6.1 |
| (6) | (Vinyl dimethicone/methicone | 10.0 | 10.0 | 10.0 | 10.0 |
| | silsesquioxane) crosspolymer*** | | | | |
| (7) | Hydrophobized boron nitride complex | 10.0 | 0.0 | 0.0 | 0.0 |
| | powder | | | | |
| (8) | Boron nitride complex powder | 0.0 | 10.0 | 0.0 | 0.0 |
| (9) | Boron nitride complex sintered | 0.0 | 0.0 | 10.0 | 0.0 |
| | powder (Production Example 3) | | | | |
| (10) | Boron nitride**** | 0.0 | 0.0 | 0.0 | 10.0 |
| (11) | Methylparaben | 0.4 | 0.4 | 0.4 | 0.4 |
| (12) | Propylparaben | 0.4 | 0.4 | 0.4 | 0.4 |
| (13) | Magnesium stearate | 1.0 | 1.0 | 1.0 | 1.0 |
| (14) | Silica | 4.0 | 4.0 | 4.0 | 4.0 |
| (15) | Octylmethoxy cinnamate | 7.5 | 7.5 | 7.5 | 7.5 |
| (16) | Glyceryl tri-2-ethylhexanoate | 6.6 | 6.6 | 6.6 | 6.6 |
| (17) | Dimethicone | 3.0 | 3.0 | 3.0 | 3.0 |
| (18) | Tocopheryl acetate | 0.5 | 0.5 | 0.5 | 0.5 |

*Perfluoroalkyl phosphate-treated (PF-treated) product; Daito Kasei Kogyo Co., Ltd.
**SP-500; Toray Industries, Inc.
***KSP-100; Shin-Etsu Chemical Co., Ltd.
****SHP7; Mizushima Ferroalloy Co., Ltd.

The below-described components (14) to (17) were mixed together, and it was added to and mixed with the mixture of components (1) to (13) which had been mixed together in advance. After that, the obtained mixture was pulverized using a hammer mill (Dalton Co., Ltd.), and weighing was performed to obtain 13 g of the pulverized mixture. It was pressed using a powder pressing machine (Sanshinseiki Co., Ltd.) to obtain a powder foundation.

TABLE 2

| | | | | Comparative |
|---|---|---|---|---|
| | | Example 3 | Example 4 | Example 3 |
| (1) | Hydrophobized titanium | 0.0 | 0.0 | 9.0 |
| | oxide* | | | |
| (2) | Hydrophobized iron oxide* | 0.0 | 0.0 | 4.0 |
| (3) | Hydrophobized talc* | 10.0 | 10.0 | 4.0 |
| (4) | Hydrophobized mica* | 13.0 | 13.0 | 6.0 |
| (5) | Hydrophobized boron | 37.0 | 0.0 | 0.0 |
| | nitride complex powder | | | |
| (6) | Boron nitride complex | 0.0 | 37.0 | 0.0 |
| | powder | | | |
| (7) | Boron nitride** | 0.0 | 0.0 | 37.0 |
| (8) | Nylon powder*** | 6.6 | 6.6 | 6.6 |
| (9) | (Vinyl dimethicone/ | 10.0 | 10.0 | 10.0 |
| | methicone silsesquioxane) | | | |
| | crosspolymer**** | | | |
| (10) | Methylparaben | 0.4 | 0.4 | 0.4 |
| (11) | Propylparaben | 0.4 | 0.4 | 0.4 |
| (12) | Magnesium stearate | 1.0 | 1.0 | 1.0 |
| (13) | Silica | 4.0 | 4.0 | 4.0 |
| (14) | Octylmethoxy cinnamate | 7.5 | 7.5 | 7.5 |
| (15) | Glyceryl tri-2- | 6.6 | 6.6 | 6.6 |
| | ethylhexanoate | | | |
| (16) | Dimethicone | 3.0 | 3.0 | 3.0 |
| (17) | Tocopheryl acetate | 0.5 | 0.5 | 0.5 |

*Perfluoroalkyl phosphate-treated (PF-treated) product; Daito Kasei Kogyo Co., Ltd.
**SHP7; Mizushima Ferroalloy Co., Ltd.
***SP-500; Toray Industries, Inc.
****KSP-100; Shin-Etsu Chemical Co., Ltd.

### [2] Liquid Foundation

The below-described components (1) to (10) were homogeneously mixed together and dispersed to prepare an oil phase. Components (11) to (15) were also mixed together and then dissolved at 70° C. The obtained mixture was then allowed to cool. (16) was then added thereto to prepare an aqueous phase. The obtained aqueous phase was slowly added to the oil phase under stirring. Finally, component (17), (18) or (19) were added thereto under stirring to be homogeneously admixed. The obtained mixture was subjected to defoaming using a simplified defoaming device in which a vacuum pump was connected to a desiccator, thereby obtaining a liquid foundation.

TABLE 3

| | | | Comparative | Comparative |
|---|---|---|---|---|
| | | Example 5 | Example 4 | Example 5 |
| (1) | PEG-10 dimethicone* | 2.0 | 2.0 | 2.0 |
| (2) | (Dimethicone/ | 1.0 | 1.0 | 1.0 |
| | (PEG-10/15)) | | | |
| | crosspolymer** | | | |
| (3) | Methyl trimethicone*** | 24.3 | 24.3 | 24.3 |
| (4) | Tocopheryl acetate | 0.1 | 0.1 | 0.1 |
| (5) | Octylmethoxy cinnamate | 7.5 | 7.5 | 7.5 |
| (6) | Polymethyl | 4.0 | 4.0 | 4.0 |
| | silsesquioxane**** | | | |
| (7) | Dimethicone | 3.0 | 3.0 | 3.0 |
| (8) | Hydrophobized titanium | 11.0 | 11.0 | 11.0 |
| | oxide***** | | | |
| (9) | Hydrophobized iron | 2.0 | 2.0 | 2.0 |
| | oxide***** | | | |
| (10) | Hydrophobized | 3.0 | 3.0 | 3.0 |
| | talc***** | | | |
| (11) | Ion-exchange water | 30.0 | 30.0 | 30.0 |
| (12) | 1,3-butylene glycol | 3.0 | 3.0 | 3.0 |
| (13) | Phenoxyethanol | 0.4 | 0.4 | 0.4 |
| (14) | Magnesium sulfate | 0.5 | 0.5 | 0.5 |
| (15) | Methylparaben | 0.2 | 0.2 | 0.2 |
| (16) | Ethanol | 3.0 | 3.0 | 3.0 |
| (17) | Hydrophobized boron | 5.0 | 0.0 | 0.0 |
| | nitride complex | | | |
| | powder | | | |
| (18) | Boron nitride complex | 0.0 | 5.0 | 0.0 |
| | sintered powder | | | |
| | (Production Example 3) | | | |
| (19) | Hydrophobized boron | 0.0 | 0.0 | 5.0 |
| | nitride | | | |

*KF-6017; Shin-Etsu Chemical Co., Ltd.
**KSG-210; Shin-Etsu Chemical Co., Ltd.
***TMF-1.5; Shin-Etsu Chemical Co., Ltd.
****Tospearl 150KA; Momentive
*****Perfluoroalkyl phosphate- and triethoxycaprylylsilane-treated (FOTS-treated) product; Daito Kasei Kogyo Co., Ltd.

### [3] Lip Stick

The below-described components (1) to (10) were homogeneously mixed together with heating at 85° C. to obtain a first mixture. Components (11) to (13) were homogeneously mixed together with further heating at 85° C. to obtain a second mixture. The second mixture was then added to the first one. The resulting mixture was cooled to room temperature, thereby obtaining a lip stick.

TABLE 4

| | | | Comparative |
|---|---|---|---|
| | | Example 6 | Example 6 |
| (1) | Bis-behenyl/isostearyl/phytosteryl dimer | 15.0 | 15.0 |
| | dilinoleyl dimer dilinoleate* | | |
| (2) | Phytosteryl/behenyl/octyldodecyl/ | 15.0 | 15.0 |
| | isostearyl lauroyl glutamate** | | |
| (3) | Diisostearyl malate*** | 15.0 | 15.0 |
| (4) | Tri(caprylic acid/capric acid) glyceryl | 10.0 | 10.0 |
| (5) | Synthetic wax | 6.0 | 6.0 |
| (6) | Ethylene-propylene copolymer | 3.0 | 3.0 |
| (7) | Squalene | 11.5 | 11.5 |
| (8) | Isotridecyl isononanoate | 13.0 | 13.0 |
| (9) | Sorbitan sesquioleate | 2.0 | 2.0 |
| (10) | Tocopheryl acetate | 0.5 | 0.5 |
| (11) | Organic lake pigment | 4.0 | 4.0 |
| (12) | Boron nitride complex powder | 5.0 | 0.0 |
| (13) | Boron nitride**** | 0.0 | 5.0 |

*Plandool-G; Nippon Fine Chemical
**Eldew PS 308; Ajinomoto Co., Inc.
***Cosmol 222 (Salacos 222); The Nisshin OilliO Group, Ltd.
****SHP7; Mizushima Ferroalloy Co., Ltd.

### [4] Mascara

The below-described components (1) to (12) were homogeneously mixed together with heating at 80° C. to obtain a first mixture. Components (13) to (18) were dissolved by heating at 85° C. to obtain a second mixture. The second mixture was then added and homogeneously mixed to the first one. The resulting mixture was cooled to room temperature, and component (19) was added thereto. After homogeneously mixing, a mascara was obtained.

TABLE 5

| | | | Comparative |
|---|---|---|---|
| | | Example 7 | Example 7 |
| (1) | Ion-exchange water | 43.90 | 43.90 |
| (2) | Bentonite* | 1.50 | 1.50 |
| (3) | Phenoxyethanol | 0.50 | 0.50 |
| (4) | Hydroxyethyl Cellulose** | 2.00 | 2.00 |
| (5) | Sericite*** | 0.50 | 0.50 |
| (6) | PEG-20 glyceryl stearate | 5.00 | 5.00 |
| (7) | 1,3-butylene glycol | 6.00 | 6.00 |
| (8) | Boron nitride complex powder | 15.00 | 0.00 |
| (9) | Boron nitride**** | 0.00 | 15.00 |
| (10) | Iron oxide***** | 2.00 | 2.00 |
| (11) | Titanium oxide | 1.00 | 1.00 |
| (12) | Triethanolamine | 1.00 | 1.00 |
| (13) | Carnauba wax | 1.00 | 1.00 |
| (14) | Bees wax | 6.50 | 6.50 |
| (15) | Cetanol | 2.00 | 2.00 |
| (16) | Stearic acid | 2.00 | 2.00 |
| (17) | Polyisobutene | 1.00 | 1.00 |
| (18) | Tocopheryl acetate | 0.10 | 0.10 |
| (19) | Acrylates copolymer | 9.00 | 9.00 |

*Kunipia-G; Kunimine Industries Co., Ltd.
**HEC Daicel SE900; Daicel Chemical Industries, Ltd.
***Whitetex; BASF
****SHP7; Mizushima Ferroalloy Co., Ltd.
*****TAROX BL-100P; Titan Kogyo, Ltd.

### 3. Assessment of Cosmetic Compositions

For each of the obtained cosmetic compositions, feeling of glow, texture and adhesion to the skin were tested using the assessment methods described below.

### [1] Feeling of Glow

### (Assessment Method)

The assessment regarding feeling of glow of each of the cosmetic compositions after applied was carried out by a panel consisting of 10 women based on the below-described assessment criteria. A cosmetic composition which provided natural glow and lively feeling but no excessive greasy feeling and no matte finish was regarded as having adequate feeling of glow.

(Assessment Criteria) ⊚: 8 or more out of 10 women recognized adequate feeling of glow. ∘: 7 out of 10 women recognized adequate feeling of glow. Δ: 4 to 6 out of 10 women recognized adequate feeling of glow. x: 3 or less out of 10 women recognized adequate feeling of glow.

### [2] Texture

### (Assessment Method)

The assessment regarding texture of each of the cosmetic compositions after applied was carried out by a panel consisting of 10 women based on the below-described assessment criteria. A cosmetic composition which was successfully applied homogeneously to the irregular facial skin surface to provide fine texture without unevenness of finish after applied was regarded as having good texture.

(Assessment Criteria) ⊚: 8 or more out of 10 women recognized good texture. ∘: 7 out of 10 women recognized good texture. Δ: 4 to 6 out of 10 women recognized good texture. x: 3 or less out of 10 women recognized good texture.

### [3] Adhesion

### (Assessment Method)

The assessment regarding adhesion of each of the cosmetic compositions after applied was carried out by a panel consisting of 10 women based on the below-described assessment criteria. A cosmetic composition which provided no deterioration and no separation after applied and was beautifully attached to the irregular facial skin surface was regarded as having good adhesion.

(Assessment Criteria) ⊚: 8 or more out of 10 women recognized good adhesion. ∘: 7 out of 10 women recognized good adhesion. Δ: 4 to 6 out of 10 women recognized good adhesion. x: 3 or less out of 10 women recognized good adhesion.

The results are shown in Table 6.

TABLE 6

| | | Feeling | | |
|---|---|---|---|---|
| | | of glow | Texture | Adhesion |
| Foundation | Example 1 | ⊚ | ⊚ | ⊚ |
| (powder) | Example 2 | ⊚ | ◯ | ⊚ |
| | Comparative Example 1 | X | X | ◯ |
| | Comparative Example 2 | Δ | X | Δ |
| Foundation | Example 3 | ⊚ | ⊚ | ◯ |
| (powder) | Example 4 | ⊚ | ⊚ | ⊚ |
| | Comparative Example 3 | X | X | Δ |
| Foundation | Example 5 | ⊚ | ⊚ | ⊚ |
| (liquid) | Comparative Example 4 | X | X | ◯ |
| | Comparative Example 5 | Δ | X | Δ |
| Lip stick | Example 6 | ⊚ | ◯ | ⊚ |
| | Comparative Example 6 | ◯ | X | Δ |
| Mascara | Example 7 | ⊚ | ⊚ | ⊚ |
| | Comparative Example 7 | Δ | X | X |

As shown in Table 6, the products of the working examples provided more adequate feeling of glow, finer texture and better adhesion compared to the products of the comparative examples. Thus, the cosmetic composition of the present invention provides a good balance between smoothness to the touch and the covering ability. In addition, when the cosmetic composition is applied to the skin, lively finish with fine texture and natural glow and adhesion to the skin can be provided. Further, in the case of the cosmetic compositions comprising the boron nitride complex sintered powder obtained in Production Example 3 shown as Comparative Examples 1 and 4, the entire skin looked dull, and it was difficult to provide natural glow. In addition, perhaps because the surface appearance of the boron nitride complex sintered powder was red-brown, many women felt that the texture was rough.

### INDUSTRIAL APPLICABILITY

The cosmetic composition comprising the boron nitride complex powder according to the present invention is suitably used, in particular, as a cosmetic composition for makeup such as foundation, eye shadow, lip stick, lip gloss, concealer, blush, mascara, eyeliner, lipliner and eyeliner pencil, or as a cosmetic composition for skincare such as emulsion, cream and sunscreen.

1. A cosmetic composition comprising a boron nitride complex powder in which particles of a metal oxide are attached to at least a part of the surface of a base material consisting of boron nitride.

2. The cosmetic composition according to claim 1, wherein the metal oxide is one or a combination of two or more substances selected from the group consisting of titanium dioxide, iron oxide, zinc oxide and chrome oxide.

3. The cosmetic composition according to claim 1, wherein the metal oxide comprises a combination of titanium dioxide, yellow iron oxide, red iron oxide and black iron oxide.

4. The cosmetic composition according to claim 3, wherein the metal oxide comprises 50 to 95.8 wt % of titanium dioxide, 3 to 35 wt % of yellow iron oxide, 1 to 11 wt % of red iron oxide and 0.2 to 4 wt % of black iron oxide based on the total weight of the metal oxide.

5. The cosmetic composition according to claim 1, wherein the average particle size of the boron nitride complex powder is 2 μm to 35 μm when measured by laser diffraction scattering method.

6. The cosmetic composition according to claim 1, wherein the average particle size of the boron nitride base material is 1 μm to 30 μm when measured by laser diffraction scattering method, and the average particle size of the particles of the metal oxide is 0.1 μm to 2 μm when measured with a scanning electron micrograph.

7. The cosmetic composition according to claim 1, wherein the weight ratio of boron nitride base material/metal oxide particle is 75/25 to 30/70.

8. The cosmetic composition according to claim 1, further comprising a component allowed to be used in cosmetics.

9. The cosmetic composition according to claim 1, which comprises 0.1 to 50 wt % of the boron nitride complex powder based on the total weight of the cosmetic composition.

10. The cosmetic composition according to claim 1, which is in the form of a makeup cosmetic or skincare cosmetic.

11. The cosmetic composition according to claim 10, which is in the form of a liquid foundation or powder foundation.

12. The cosmetic composition according to claim 10, which is in the form of a lipstick, eye shadow, eye liner or mascara.

13. The cosmetic composition according to claim 2, wherein the metal oxide comprises a combination of titanium dioxide, yellow iron oxide, red iron oxide and black iron oxide.

14. The cosmetic composition according to claim 2, wherein the average particle size of the boron nitride complex powder is 2 μm to 35 μm when measured by laser diffraction scattering method.

15. The cosmetic composition according to claim 3, wherein the average particle size of the boron nitride complex powder is 2 μm to 35 μm when measured by laser diffraction scattering method.

16. The cosmetic composition according to claim 2, wherein the average particle size of the boron nitride base material is 1 μm to 30 μm when measured by laser diffraction scattering method, and the average particle size of the particles of the metal oxide is 0.1 μm to 2 μm when measured with a scanning electron micrograph.

17. The cosmetic composition according to claim 3, wherein the average particle size of the boron nitride base material is 1 μm to 30 μm when measured by laser diffraction scattering method, and the average particle size of the particles of the metal oxide is 0.1 μm to 2 μm when measured with a scanning electron micrograph.

18. The cosmetic composition according to claim 2, wherein the weight ratio of boron nitride base material/metal oxide particle is 75/25 to 30/70.

19. The cosmetic composition according to claim 3, wherein the weight ratio of boron nitride base material/metal oxide particle is 75/25 to 30/70.

20. The cosmetic composition according to claim 2, which comprises 0.1 to 50 wt % of the boron nitride complex powder based on the total weight of the cosmetic composition.
